(19) United States
(12) Patent Application Publication
(10) Pub. No.: US 2026/0066100 A1
(43) Pub. Date: Mar. 5, 2026

(54) DEVICE AND METHOD FOR ORGAN POSITIONING ASSESSMENT

(71) Applicant: Siemens Healthineers AG, Forchheim (DE)

(72) Inventors: Ramyar BINIAZAN, Nuernberg (DE); Andreas FIESELMANN, Erlangen (DE)

(73) Assignee: Siemens Healthineers AG, Forchheim (DE)

(21) Appl. No.: 19/311,383

(22) Filed: Aug. 27, 2025

(30) Foreign Application Priority Data
Aug. 28, 2024 (EP) 24197014.4

(51) Int. Cl. G16H 30/40; G16H 30/20

(52) U.S. Cl. CPC G16H 30/40; G16H 30/20

(57) ABSTRACT

A device for organ positioning assessment for acquiring images in the course of a medical examination of a patient, comprises: a data-interface configured to receive and/or automatically retrieve physical patient-data; a pre-assessment-unit configured to generate a positioning-advice for the medical examination based on the type of medical examination and the patient-data; a data-interface configured to output the positioning-advice; a data-interface configured to receive and/or automatically retrieve a number of examination-images of the patient; a post-assessment-unit configured to generate patient-specific feedback-data based at least on the number of examination-images and the positioning-advice; and a data-interface configured to output the feedback-data.

### CROSS-REFERENCE TO RELATED APPLICATION(S)

The present application claims priority under 35 U.S.C. § 119 to European Patent Application No. 24197014.4, filed Aug. 28, 2024, the entire contents of which are incorporated herein by reference.

### FIELD

One or more example embodiments of the present invention describe a device and a method for organ positioning assessment for the acquisition of images in the course of a medical examination of a patient as well as a medical imaging system.

### BACKGROUND

Correct organ positioning is crucial for many kinds of medical examinations. For example, in order to receive optimal X-ray mammograms, the breast must be positioning correctly. However, also for other examinations, e.g. of the chest, the knee or the head, the respective body-part must be arranged correctly, in order to have a good view on the region of interest.

Looking at the exemplary examination of breast cancer screening, the positioning of the breast must follow certain established guidelines. If the breast is not positioned well, the mammogram may not be suitable for diagnostic purposes. However, an ideal breast positioning (according to the general guideline) may not always be feasible due to patient-individual constraints, e.g. body weight, disabilities or physical constraints.

A technical problem appears that there is currently only a general support but no patient-specific (e.g. individual constraints or physical inabilities) and/or radiographer-specific (e.g. the level of experience) positioning support available for a mammographic examination. This also appears for other kinds of examinations.

Current solutions for assessment of breast positioning (e.g. TruPGMI, Volpara Health) are based on analyzing the just acquired mammography images. However, such solutions have limitations. They assess each image based on a general guideline and they don't consider patient constraints and also not the radiographers' level of experience. In addition, they can only be used after acquisition, therefore possible mispositioning cannot be avoided.

### SUMMARY

It is an object of one or more example embodiments of the present invention to improve the known systems and methods and provide a device and a method for organ positioning assessment for the acquisition of images in the course of a medical examination of a patient as well as a medical imaging system, for overcoming the above described problems. Especially, an object of one or more example embodiments of the present invention is to give a radiographer the optimal information for correct patient positioning.

At least this object is achieved by a device, a method and/or a medical imaging system according to the independent claims.

A device according to one or more example embodiments of the present invention serves for organ positioning assessment for the acquisition of images in the course of a medical examination of a patient. It comprises:
a data-interface designed for receiving and/or automatically retrieving physical patient-data,
a pre-assessment-unit designed for generating a positioning-advice for the medical examination based on the type of medical examination and the provided patient-data,
a data-interface designed for outputting the positioning-advice,
a data-interface designed for receiving and/or automatically retrieving a number of examination-images of the patient,
a post-assessment-unit designed for generating patient-specific feedback-data based at least on the number of examination-images and the positioning-advice
a data-interface designed for outputting the feedback-data.

One or more example embodiments of the present invention deal with organ positioning assessment for the acquisition of images in the course of a medical examination of a patient. It is very advantageous for X-ray imaging, e.g. fluoroscopy or radiography, but also for MRT-examinations or ultrasound examinations.

The device comprises a pre-assessment-unit, a post-assessment-unit and a number of data-interfaces that could be multiple data-interfaces or one single data-interface with multiple functionalities or multiple connections. There could be a data-interface for receiving data and another data-interface for sending data or there could be a data-interface for the pre-assessment-unit and another data-interface for the post-assessment-unit.

The (or one) data-interface is designed for receiving and/or automatically retrieving physical patient-data. This physical patient-data could include patient-data about physical inabilities or malformations of the patient, but also physical issues concerning prior exams of this patient, e.g. re-positioning, special positioning.

The pre-assessment-unit is designed for generating a positioning-advice for the medical examination based on the type of medical examination and the provided patient-data. The positioning-advice may be an advice for the required body posture of the patient as well as information about what special issues should be respected (e.g. “caution: broken arm”).

Since this advice is individual for different kinds of examinations, the positioning-advice depends on the type of medical examination (e.g. mammography, examination of the chest or knee). Furthermore, the positioning-advice depends on abilities and disabilities of the patient.

Thus, the positioning-advice is also based on the provided patient-data. In the case the patient has a special physical inability (e.g. wheelchair bound), this has to be respected for the applicable and possible body posture.

The (or one) data-interface is designed for outputting the positioning-advice. This could be done on a display, especially graphically, but also in form of control commands to control motors of an automatic positioning device. The position advice could comprise information about a center position of the body in addition with rotation angles around body axes and/or the position of extremities relative to a body-region.

These units are used for a first analysis (and possibly for positioning) before the actual examination. The following units are used for a second analysis and for producing feedback information.

The (or one) data-interface is designed for receiving and/or automatically retrieving a number of examination-images of the patient. These examination-images could be directly acquired during an examination or downloaded from a database after the examination. These examination-images are preferably recorded with the positioning-advice given by the units explained above. However, they could e.g. also be recorded in order to check other positioning-advices.

The post-assessment-unit is designed for generating patient-specific feedback-data based at least on the number of examination-images and the positioning-advice (and especially also the patient-data). The feedback-data should give a feedback based on the quality of the image about the positioning advice. This feedback-data could be used to ameliorate the device (in order to train machine-learning models used by the device), to train a user or as background information for an examiner. The feedback-data could comprise information about how a positioning-advice has been implemented, about the quality of a positioning-advice or for comparison of positioning-advices. It is preferred to additionally use the patient data in order to evaluate the condition of the positioning-advice for the feedback data. For example, when a patient is wheelchair-bound, the last positioning-advices could be used in order to evaluate whether the images of the actual positioning-advice are better or not compared to the images recorded with other positioning-advices.

The (or one) data-interface is designed for outputting the feedback-data. This could be displayed to a user, used for training or for evaluation issues.

A method according to one or more example embodiments of the present invention serves for organ positioning assessment for the acquisition of images in the course of a medical examination of a patient, especially with a device according to one or more example embodiments of the present invention. It comprises the following steps:
providing physical patient-data about the patient and/or prior exams of this patient,
generating a positioning-advice for the medical examination based on the type of medical examination and the provided patient-data,
outputting the positioning-advice and using it to position the patient for the medical examination and acquire a number of examination-images of the patient,
providing an examination-image of the patient,
generating patient-specific feedback-data based at least on the number of examination-images and the positioning-advice,
outputting the feedback-data.

First, physical patient-data about the patient and/or prior exams of this patient are provided. As already indicated above, this data should give information about the physical abilities of the patient concerning the examination. For example, the data may give at least information about the body part to be examined (e.g. known condition of a breast for mammography), but preferably also about the patient (size, weight, age). It is preferred that the physical data comprises information about inabilities (e.g. “wheelchair bound”, “broken arm”, “uncooperative”, “panics in small rooms”). However, also data about prior exams could be very helpful, like positioning for these exams or failures due to mispositioning. In short, any information that could be used for an (optimal) positioning of the patient could be included in the physical data.

The positioning-advice is preferably generated automatically. This could be done by a machine-learning model trained with physical data of the patient and information about the examination and an information about the optimal positioning as ground truth. However, this could also be done by a conventional algorithm that links keywords of the patient data with positions given in a list for a certain type of examination. There could be several lists for several types of examination.

As already said above, this positioning-advice is outputted (e.g. on a screen for a user or as control commands for an automatic positioning system) and used it to position the patient for the medical examination and acquire a number of examination-images of the patient.

Then the images are acquired. This well-known step is not inadequately needed for the method, but the images acquired should be used. However, it is not necessary to position the patient as suggested by the method. Since the user is free to follow the positioning advice or not, it could also be examined what happens, if a user should ignore the suggestions of the method. It follows the second analysis producing a feedback.

The first step of this second analysis is the provision of an examination-image of the patient, preferably the examination-images where the positioning advice has been followed.

Now, patient-specific feedback-data is generated based at least on the number of examination-images and the positioning-advice (and especially also the patient-data). This could be done in an automatic manner by an algorithm designed to search the examination-images for special structures and compare the structures with a given list of structures. In the case all structures have been found, the feedback is good, in the case there are structures missing or deformed, the feedback is not good. The search may be done by a machine-learning model trained to segment images and search for structures in these segments.

Then, the feedback-data is outputted as already explained above.

In a practical approach the method may be realized as explained in the following. The device or the method provides advice and feedback to a radiographer about positioning, e.g. of a breast, at two time points: prior and after an X-ray examination. The system uses available data from the patient which comprises at least one of the following information:
data about prior exams, especially mammograms, (e.g. retrieved from PACS) including their radiology reports,
general patient information (such as age, BMI/weight, disabilities; retrieved from the hospital information system for example),
situation information from the examination room (this could be a room camera for example, that records information about the patient's compliance during the examination).

At the first time point (prior to the exam), the available patient-data about the patient is used to provide the radiographer with patient-specific positioning advice. At the second time point (after the exam), the just acquired image and the available data are used to provide the radiographer with feedback. This feedback can include an assessment of the just acquired images, especially mammogram, (is there any positioning deficiency that could have been avoided?) and—if a positioning deficiency has been found—it can also include feedback for the radiographer on how to avoid this deficiency in the future.

Assuming all physical patient-data has been retrieved, several analyses can be made to give advice to the radiographer for (e.g. breast) positioning before the examination. These analyses can include:

Parsing of radiology reports (did the radiologists mention non-optimal positioning in the past?)

Analyzing all available information about the patient (BMI/weight, disabilities, . . . ) and comparing those to a database of known positioning challenges

Analyzing (possibility / willingness of) compliance of the patient based on room camera video recording.

In addition, the pixel data of prior images, e.g. mammograms, can be analyzed. There are state of the art methods to assess patient positioning according to general guidelines on mammographic images, see Watanabe et al. “Quality control system for mammographic breast positioning using deep learning”, Scientific Reports volume 13, Article number: 7066; 2023) for an example.

In the special case that when the prior images, e.g. mammograms, have been acquired by the same radiographer that is going to take the current image, then the analysis of the prior mammogram's pixel data could be used to give radiographer-specific advice.

The system may identify a mispositioning in the current acquired image. However, it is known from clinical reality that some kinds of mispositioning are difficult to avoid in a current exam situation.

There could be cases where optimal positioning is not possible, e.g. for a mammography where perfect positioning (according to the definitions in the guideline) is impossible due to surgery and having surgical clips on breast. In such cases having comparable breast size between two views is not possible. Furthermore, it could be possible that due to the pain of the patient, avoiding skin folds is also not possible.

The present invention could also identify cases of mispositioning that are not avoidable. The term “avoidable” means that this mispositioning would not be present when another person skilled in using state of the art breast positioning techniques would have made the exam.

Formally, avoidability X can be defined as X=percentage of radiographers of a reference group that can perform the positioning without the deficiency present.

The avoidability X can range from 0% (no radiographer would be able to avoid it) to 100% (all radiographers from the reference group would avoid it). It could then be classified into certain classes (or bins), for example:
0% <=X<=5% means almost impossible to avoid
5% <X<=25% difficult to avoid
25% <X<=75% generally avoidable
75% <X<=100% clearly avoidable.

A method to automatically quantify avoidability can be used to make a supervised training of a machine-learning model. During training the model takes the (current) image's pixel data and meta-information as input and is trained by radiographers' expert opinion as output.

When a deficiency has been found that was classified as at least generally avoidable then the software can provide the radiographer with detailed feedback on how to avoid such deficiencies in the future. This information can come from a well-curated and edited database of domain expert's opinion on how to avoid certain types of positioning deficiencies.

A medical imaging system according to one or more example embodiments of the present invention is especially a mammography-system or a fluoroscopy-system, and comprises a device according to one or more example embodiments of the present invention and/or it is designed for performing a method according to one or more example embodiments of the present invention.

Some units or modules of one or more example embodiments of the present invention mentioned above can be completely or partially realized as software modules running on a processor of a computing system. A realization largely in the form of software modules can have the advantage that applications already installed on an existing computing system can be updated, with relatively little effort, to install and run these units of the present application. The object of one or more example embodiments of the present invention is also achieved by a non-transitory computer program product with a computer program that is directly loadable into the memory of a computing system, and which comprises program units to perform the steps of the methods, at least those steps that could be executed by a computer, when the program is executed by the computing system. In addition to the computer program, such a computer program product can also comprise further parts such as documentation and/or additional components, also hardware components such as a hardware key (dongle etc.) to facilitate access to the software.

A non-transitory computer readable medium such as a memory stick, a hard-disk or other transportable or permanently-installed carrier can serve to transport and/or to store the executable parts of the computer program product so that these can be read from a processor unit of a computing system. A processor unit can comprise one or more microprocessors or their equivalents.

Particularly advantageous embodiments and features of the present invention are given by the dependent claims, as revealed in the following description. Features of different claim categories may be combined as appropriate to give further embodiments not described herein.

A preferred embodiment of the device comprises a classification-unit, preferably as part of the post-assessment-unit, wherein the classification-unit is designed to determine a mispositioning of the patient in the number of examination-images, and to determine, whether the mispositioning was avoidable or not.

Here it should be noted that mispositioning is basically a state. This state is quantified by the displacement for example. So avoidability refers to the state in the first place but could also be linked to the value that is a measure for the state. The term “avoidable” means that this mispositioning would not be present when another person skilled in the art (e.g. of breast positioning techniques) would have made the exam. For example, regarding a lateral mispositioning avoidability could be defined by the grade of mispositioning. A mispositioning of a mm could then be inavoidable, a mispositioning of a cm could be difficult to avoid but a mispositioning of a dm would be avoidable. But there may also be avoidabilities that could not be given by measures, for example, confuse left with right would be generally avoidable.

In general, by using an AI model, of course this is rather task-specific. A preferred approach is creating a data set containing images, e.g. mammograms, with certain positioning deficiencies and expert annotations that say based on the expert's opinion if that deficiency could have been avoided by proper positioning given by the state of the art.

It is preferred that the classification-unit is a machine-learning model trained to segment regions in the number of examination-images that show irregularities due to mispositioning and allocate the segmented regions to a list of multiple mispositioning-types. It could e.g. be a mispositioning if all the desired landmarks or structures could not be seen in the images.

A preferred embodiment of the device wherein the classification-unit is designed to parse image-data of earlier examinations and determine an avoidability-value for a mispositioning based on an output of a machine-learning model trained on experts' opinions or on a ratio of occurrences of a number of mispositioning-types of the list compared to the total number of images. Instead of parsing image data, this data could also be parsed in an early stage by another unit. In this case, the device could comprise a list of avoidability-values for certain mispositioning-types.

Preferably, the classification unit is designed to bin the avoidability-values of a number of mispositioning-types into certain predefined value-bins in order to allocate avoidability-statements to the mispositioning-types based on the bins. Such statements could be: “almost impossible to avoid”, “difficult to avoid”, “generally avoidable” and “clearly avoidable”.

A preferred embodiment of the device of the preceding claims, wherein the data-interface designed for receiving and/or automatically retrieving physical patient-data and/or prior exams of this patient is designed to receive data via a Picture Archiving and Communication System PACS and/or a hospital information system HIS and/or a Radiology Information System RIS and/or sensors, especially cameras.

Regarding a preferred method, the patient-data is general patient information, such as e.g. age, BMI/weight, disabilities, alterations of organs like clips in a breast. The general patient information may be retrieved from the hospital information system (HIS) or being manually inputted. Alternatively or additionally it is preferred that the patient-data of a patient comprises data about prior exams of this patient, especially data about prior examinations similar to the intended medical examination and/or situation information from the examination room. This could be a room camera for example, that records information about the patient's compliance during the examination.

Regarding a preferred method, for generating the patient-specific positioning-advice the patient-data is analyzed, wherein this analysis includes one or more of the steps:
parsing of radiology reports, e.g. based on the question “did the radiologists mention non-optimal positioning in the past?”,
analyzing general patient information (e.g. BMI/weight, disabilities or unwillingness) and comparing those to a database of known positioning challenges,
analyzing compliance of the patient based on room camera video recording,
analyzing pixel data of images of prior exams of the patient, (see above mentioned state of the art methods).

Preferably it is determined, whether the person performing the intended medical examination has performed a similar examination on the patient and a person specific advice is generated based on the respective prior examination and added to the positioning-advice.

Thus, there is the possibility to analyze the history of the patient in order to get an overview about the optimal positioning, as well as the possibility to analyze the history of the technician performing the examination in order to get an overview about the optimal explanations for positioning.

Preferably, the intended examination is a mammography or a fluoroscopy or a CT-examination or an MRI-examination or a radiography examination or an ultrasound examination. The positioning method may be used for several different examinations that may not always be X-ray examinations. However, since X-ray examinations are always connected with a dose applied to a patient, the method is especially advantageous to prevent defective X-ray images.

According to a preferred method, for creating the feedback-data the number of examination-images is automatically assessed including concerning the quality of positioning.

The assessment preferably comprises a search for a number and/or a grade of positioning deficiencies. It is preferred that the feedback-data also includes automatically generated notes concerning methods how to avoid a positioning deficiency in the future.

Preferably, the number of examination-images is analyzed by looking for a mispositioning, determining whether the mispositioning was avoidable or not. As said above, the term “avoidable” means that this mispositioning would not be present when another person skilled in using state of the art breast positioning techniques would have made the exam.

It is preferred that an avoidability-value is calculated as a percentage of radiographers that can perform the positioning without the mispositioning. Here as said above, the avoidability can be from 0% (no radiographer would be able to avoid it) to 100% (all radiographers from the group would avoid it) and it could then be classified into several classes.

Preferably, avoidability-values of a number of mispositioning-types are binned into certain predefined value bins in order to allocate avoidability-states to the mispositioning-types based on the bins. These states may be “almost impossible to avoid”, “difficult to avoid”, “generally avoidable”, “clearly avoidable”.

Regarding a preferred method, where a mispositioning is found in an examination image, the method comprises the steps:
assigning the mispositioning in the examination-image to a mispositioning-type of a predefined list,
determining an avoidability for this mispositioning type based on an avoidability-value or an avoidability-statement,
comparing the avoidability with a predefined threshold-value indicating whether the mispositioning-type is avoidable or not,
in the case the comparison shows that the mispositioning-type is avoidable, outputting a predefined dataset allocated to the mispositioning type comprising detailed information on how to avoid such mispositioning in the future. This information may come from a well-curated and edited database of domain expert's opinion on how to avoid certain types of positioning deficiencies.

Preferably, the positioning-advice is designed according to the physical nature of the patient, and is especially patient-specific, and/or is designed based on predefined information and/or prior data of the examiner for the medical examination.

The use of AI-based methods (AI: “artificial intelligence”) is preferred for the method according to one or more example embodiments of the present invention. Artificial intelligence is based on the principle of machine-based learning and is usually carried out with an adaptive algorithm that has been trained accordingly. The expression “machine-learning” is often used for machine-based learning, which also includes the principle of “deep learning”.

The methods may also include elements of “cloud computing”. In the technical field of “cloud computing”, an IT infrastructure is provided over a data-network, storage space or processing power and/or application software. The communication between the user and the “cloud” is achieved via data-interfaces and/or data transmission protocols. In the context of “cloud computing”, in a preferred embodiment of the methods according to the present invention, provision of data via a data channel (for example a data-network) to a “cloud” takes place. This “cloud” includes a (remote) computing system, e.g. a computer cluster that typically does not include the user's local machine. It is particularly preferred that the cloud service provides as well computing power as application software.

### BRIEF DESCRIPTION OF THE DRAWINGS

Other objects and features of the present invention will become apparent from the following detailed descriptions considered in conjunction with the accompanying drawings. It is to be understood, however, that the drawings are designed solely for the purposes of illustration and not as a definition of the limits of the present invention.

FIG. 1 shows a tomography-system (as example for an imaging system),

FIG. 2 shows an example for an advice according to one or more example embodiments of the present invention,

FIG. 3 shows a block diagram of the method according to one or more example embodiments of the present invention,

FIG. 4 shows a practical application of one or more example embodiments of the present invention.

### DETAILED DESCRIPTION

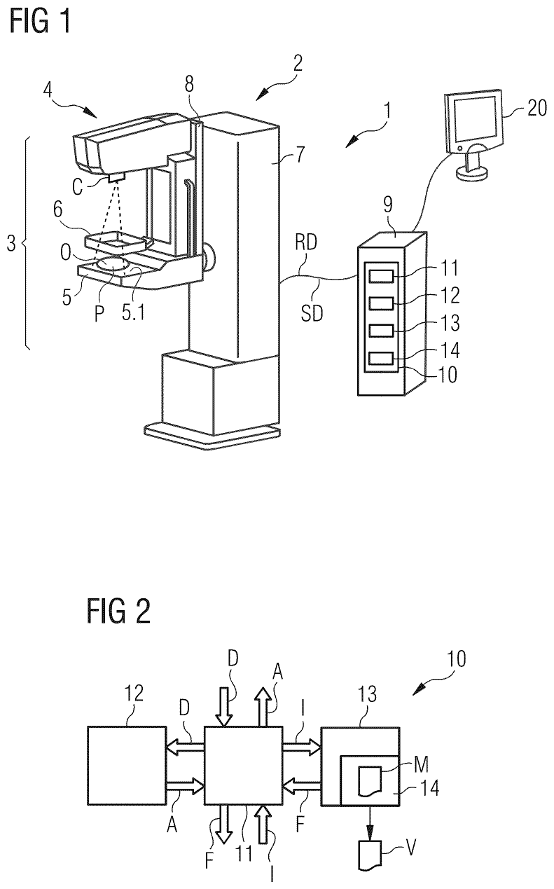

In FIG. 1, a mammography system 1 in the form of a tomosynthesis system 1 is roughly schematically shown as an example for a medical imaging system. Relative directional information such as “top”, “bottom” etc. refers to a tomosynthesis system 1 that has been set up for operation as intended. The tomosynthesis system 1 includes a tomosynthesis device 2 and a control device 9.

The tomosynthesis device 2 has a column 7 and a source-detector arrangement 3, which in turn include an X-ray emitter 4 and a detector 5 with a detector surface 5.1. The column 7 stands on the ground during operation. The source-detector arrangement 3 is slidably connected to it, so that the height of the detector surface 5.1, i.e. the distance to the ground, can be adjusted to a patient's chest height.

A breast O of the patient (shown here schematically) lies on the top side of the detector surface 5.1 as an examination object O to be examined. A compression plate 6 is arranged above the breast O and the detector surface 5.1, which is slidably connected to the source-detector arrangement 3. For the examination, the breast O is compressed and at the same time fixed by lowering the compression plate 6 onto it, so that pressure is exerted on the breast O between the compression plate 6 and the detector surface 5.1.

The X-ray emitter 4 is arranged and designed opposite the detector 5 in such a way that the detector 5 detects X-ray radiation R emitted by it after at least part of the X-ray radiation R has penetrated the patient's breast O. The X-ray emitter 4 can be pivoted relative to the detector 5 via a rotating arm 8 in a range of ±50° about a basic position in which it is vertically above the detector surface 5.1. The section to be recorded can be specified or restricted using a collimator C.

The control device 9 receives the raw data RD of the measurement and sends control data SD to the tomosynthesis device 2 using a data interface. It is connected to a terminal 20 through which a user can communicate commands to the tomosynthesis system 1 or retrieve measurement results. The control device 9 can be arranged in the same room as the tomosynthesis device 2, but it can also be located in an adjacent control room or at an even further spatial distance

The control device 9 comprises a device 10 for organ positioning assessment for the acquisition of images I in the course of a medical examination of a patient P. The device comprises a data-interface 11, a pre-assessment-unit 12, a post-assessment-unit 13 and classification-unit 14.

FIG. 2 shows an example for an advice according to one or more example embodiments of the present invention.

The data-interface 11 is designed for receiving and/or automatically retrieving physical patient-data D and outputting the positioning-advice A. Furthermore, the data-interface 11 is designed for receiving and/or automatically retrieving a number of examination-images I of the patient P and outputting the feedback-data F. The data-interface 11 could be designed for receiving and/or automatically retrieving physical patient-data D and/or prior exams of this patient P is designed to receive data via a Picture Archiving and Communication System PACS and/or a hospital information system HIS and/or a Radiology Information System RIS and/or sensors, especially cameras.

The pre-assessment-unit 12 is designed for generating a positioning-advice A for the medical examination based on the type of medical examination and the provided patient-data D.

The post-assessment-unit 13 is designed for generating patient-specific feedback-data F based at least on the number of examination-images I and the positioning-advice A.

The classification-unit 14 is designed to determine a mispositioning of the patient P in the number of examination-images I, and to determine, whether the mispositioning was avoidable or not. The classification-unit 14 may be a machine-learning model trained to segment regions in the number of examination-images I that show irregularities due to mispositioning and allocate the segmented regions to a list of multiple mispositioning-types M. The classification-unit 14 could be designed to parse image-data I of earlier examinations and determine an avoidability-value V for a mispositioning based on an output of a machine-learning model trained on experts' opinions or on a ratio of occurrences of a number of mispositioning-types M of the list compared to the total number of images I. The classification unit could also be designed to bin the avoidability-values V of a number of mispositioning-types M into certain predefined value-bins in order to allocate avoidability-statements to the mispositioning-types M based on the bins.

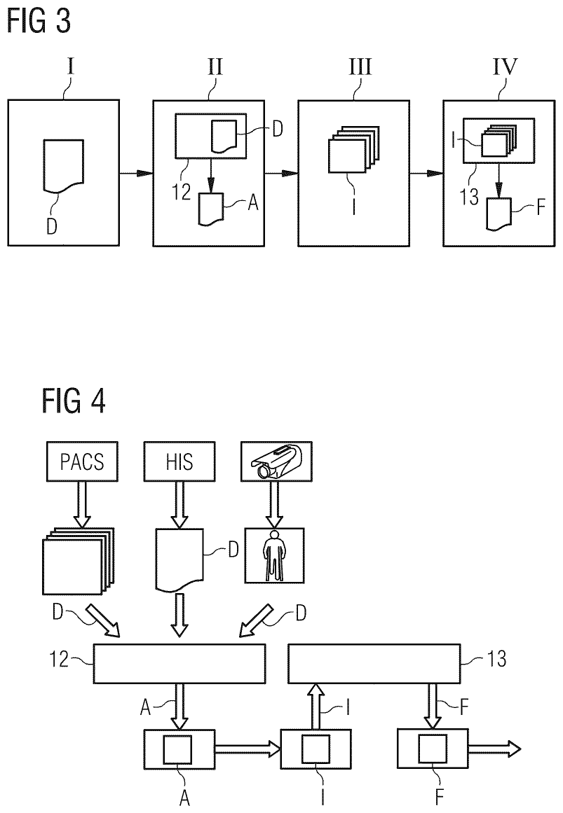

FIG. 3 shows a block diagram of the method for organ positioning assessment for the acquisition of images I in the course of a medical examination of a patient P, especially with a device 10 as shown in FIG. 2.

In step I, physical patient-data D and/or prior exams of this patient P is provided.

In step II, a positioning-advice A for the medical examination is generated by the pre-assessment-unit 12 based on the type of medical examination and the provided patient-data D and outputted in order to use it to position the patient P for the medical examination and acquire a number of examination-images I of the patient P.

I step III, an examination-image I of the patient P is provided, e.g. after acquisition.

In step IV, patient-specific feedback-data F is generated by the post-assessment-unit 13 based at least on the number of examination-images I and the positioning-advice A and outputted.

FIG. 4 shows a practical application of one or more example embodiments of the present invention. From above, physical patient-data D about the patient P (middle) and/or prior exams of this patient P (left) or the actual condition recorded with a camera (right) is provided.

This data is inputted in a pre-assessment-unit 12 and a positioning-advice A for the medical examination is generated and outputted (middle row).

The bottom row shows that the positioning-advice A is used to make an exam and acquire images I.

These images I are then inputted in a post-assessment-unit 13 and patient-specific feedback-data F is generated and outputted.

Although the present invention has been disclosed in the form of preferred embodiments and variations thereon, it will be understood that numerous additional modifications and variations could be made thereto without departing from the scope of the present invention. For the sake of clarity, it is to be understood that the use of “a” or “an” throughout this application does not exclude a plurality, and “comprising” does not exclude other steps or elements. The expression “a number of” means “at least one”. The mention of a “unit” or a “device” does not preclude the use of more than one unit or device. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A device for organ positioning assessment for acquiring images during a medical examination of a patient, the device comprising:
a first data-interface configured to at least one of receive or automatically retrieve physical patient-data;
a pre-assessment-unit configured to generate a positioning-advice for the medical examination based on a type of the medical examination and the physical patient-data;
a second data-interface configured to output the positioning-advice;
a third data-interface configured to at least one of receive or automatically retrieve a number of examination-images of the patient;
a post-assessment-unit configured to generate patient-specific feedback-data based at least on the number of examination-images and the positioning-advice; and
a fourth data-interface configured to output the patient-specific feedback-data.

2. The device according to claim 1, further comprising:
a classification-unit configured to determine a mispositioning of the patient in the number of examination-images; and
determine whether the mispositioning was avoidable or not.

3. The device according to claim 2, wherein the classification-unit is configured to parse image-data of earlier examinations and determine an avoidability-value for the mispositioning based on an output of a machine-learning model trained on experts' opinions or on a ratio of occurrences of a number of mispositioning-types compared to a total number of images.

4. The device according to claim 1, wherein the first data-interface is configured to receive the physical patient-data via at least one of a Picture Archiving and Communication System, a hospital information system, a Radiology Information System, or sensors.

5. A method for organ positioning assessment for acquiring images during a medical examination of a patient, the method comprising:
providing physical patient-data about at least one of the patient or prior exams of the patient;
generating a positioning-advice for the medical examination based on a type of the medical examination and the physical patient-data;
outputting and using the positioning-advice to position the patient for the medical examination and to acquire a number of examination-images of the patient;
providing an examination-image of the patient;
generating patient-specific feedback-data based at least on the number of examination-images and the positioning-advice; and
outputting the patient-specific feedback-data.

6. The method according to claim 5, wherein at least one of
the physical patient-data is general patient information, or
the physical patient-data includes data about prior examinations of the patient.

7. The method according to claim 5, wherein
the generating of the positioning-advice includes analyzing the physical patient-data, and the analyzing includes
parsing of radiology reports,
analyzing general patient information and comparing the general patient information to a database of known positioning challenges,
analyzing compliance of the patient based on a room camera video recording, and
analyzing pixel data of images of prior exams of the patient.

8. The method according to claim 5, wherein the medical examination is a mammography, a fluoroscopy, a CT-examination, an MRI-examination, a radiography examination or an ultrasound examination.

9. The method according to claim 5, wherein the generating of the patient-specific feedback-data comprises:
automatically assessing the number of examination-images including with regard to a quality of positioning.

10. The method according to claim 5, wherein the number of examination-images is analyzed by
looking for a mispositioning, and
determining whether the mispositioning was avoidable or not.

11. The method according to claim 10, wherein, in response to finding the mispositioning in an examination image, the method further includes:
assigning the mispositioning in the examination-image to a mispositioning-type from a list;
determining an avoidability for the mispositioning-type based on an avoidability-value or an avoidability-statement;
comparing the avoidability with a threshold-value indicating whether the mispositioning-type is avoidable or not; and
in case the comparing shows that the mispositioning-type is avoidable, outputting a dataset allocated to the mispositioning-type, the dataset including detailed information on how to avoid a future occurrence of the mispositioning.

12. The method according to claim 11, wherein the positioning-advice is configured according to at least one of
a physical nature of the patient and the positioning-advice is patient-specific, or
based on at least one of information or prior data of an examiner for the medical examination.

13. A medical imaging system comprising the device according to claim 1.

14. A non-transitory computer program product comprising a computer program that, when executed by a computer, causes the computer to carry out the method of claim 5.

15. A non-transitory computer-readable storage medium storing instructions that, when executed by a computer, cause the computer to carry out the method of claim 5.

16. The device of claim 2, wherein the classification-unit is part of the post-assessment-unit.

17. The device of claim 2, wherein the classification-unit is a machine-learning model trained to
segment regions in the number of examination-images that show irregularities due to mispositioning, and
allocate the regions to a list of multiple mispositioning-types.

18. The device of claim 3, wherein the classification-unit is configured to bin avoidability-values of the number of mispositioning-types into value-bins to allocate avoidability-statements to the number of mispositioning-types based on the value-bins.

19. The device of claim 4, wherein the sensors are cameras.

20. The method of claim 6, wherein the prior examinations are similar to at least one of the medical examination or situation information from an examination room.

21. The method of claim 7, further comprising:
determining whether a person performing the medical examination has performed a prior similar examination on the patient;
generating person specific advice based on the prior similar examination; and
adding the person specific advice to the positioning-advice.

22. The method of claim 9, wherein
the automatically assessing includes searching for at least one of a number or a grade of positioning deficiencies, and
the patient-specific feedback-data includes automatically generated notes concerning methods for how to avoid a future positioning deficiency.

23. The method of claim 10, wherein
an avoidability-value is calculated as a percentage of radiographers able to perform the positioning without the mispositioning,
avoidability-values of a number of mispositioning-types are binned into value bins in order to allocate avoidability-states to the number of mispositioning-types based on the value bins, and
an avoidability-value for a mispositioning is based on an output of a machine-learning model trained on experts' opinions or on a ratio of occurrences of the number of mispositioning-types compared to a total number of images.

24. The medical imaging system of claim 13, wherein the medical imaging system is a mammography-system or a fluoroscopy-system.

25. A medical imaging system configured to perform the method according to claim 5.
